(19) United States
(12) Patent Application Publication
(10) Pub. No.: US 2009/0074922 A1
(43) Pub. Date: Mar. 19, 2009

(54) METHOD AND APPARATUS FOR SANITIZING AND PROCESSING PERISHABLE GOODS IN ENCLOSED CONDUITS

(75) Inventor: Anthony J.M. Garwood, Mercer Island, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SAFEFRESH TECHNOLOGIES, LLC, Mercer Island, WA (US)

(21) Appl. No.: 12/248,386

(22) Filed: Oct. 9, 2008

Related U.S. Application Data
(63) Continuation of application No. 10/418,558, filed on Apr. 16, 2003, now abandoned.
(60) Provisional application No. 60/373,232, filed on Apr. 16, 2002.

(51) Int. Cl. G01N 33/12; A23L 3/34; A61L 2/16

(52) U.S. Cl. 426/231; 426/335; 422/255; 426/312

(57) ABSTRACT

The invention is directed to a method and apparatus for sanitizing perishable goods by mixing the goods with sanitizing liquor for a suitable period of time followed by separating the liquor and substantially neutralizing any residual sanitizing agent left in the goods. In one instance, the sanitizing agent includes ozone and water; therefore, separation of the ozonated water advantageously proceeds with a squeezing effect to more adequately remove the ozonated water from the goods.

### CROSS-REFERENCES TO RELATED APPLICATIONS

This application is a continuation of application Ser. No. 10/418,558, filed Apr. 16, 2003, which claims the benefit of Provisional Application No. 60/373,232, filed Apr. 16, 2002, both of which applications are herein expressly incorporated by reference.

### FIELD OF THE INVENTION

The present invention relates to the sanitizing, decontaminating, analyzing, proportioning, grinding and blending of perishable food items in enclosed conduits.

### BACKGROUND

Ozone has been recognized as safe to use in food processing. Accordingly, food processors have begun to use ozone in washing various foods. One processor has developed a process that utilizes ozone in the treatment of poultry. In this system, ozonated water is sprayed on the food products as the products pass through ozonated water sprays on a conveyor system. A pump moves water from the chiller bath through a filter. The filtered ozonated water is then titrated with ozone gas, effectively killing any pathogens, such as E coli 0157:H7, Listeria, and salmonella, and oxidizes any residual organic materials before being recycled through the process, thus saving on wastewater treatment costs.

A turkey processor also uses ozone to enable the recycling of process wash water. Once the water has been used, the water passes through a series of ozone vessels. Ozone gas is pumped into the vessels to kill any microorganisms. The system strips out any residual ozone prior to returning the water to a chiller. Any residual ozone is captured and run through a catalytic destruction unit. This provides for up to about 80% of recycled water, thus saving the company water, energy, and wastewater treatment costs.

However, the prior art methods for using ozonated water to wash food products are, for the most part, conducted in open vats or in ambient environments wherein the amount of ozone exposure is relatively uncontrolled.

Ozonated water remains a viable method of sanitizing or decontaminating meat or any other perishable good. However, widespread use of ozone has been hampered by the inability to properly control the amount of ozone's exposure to the meat. Ozone is a strong oxidizer and will render perishable goods, such as meat, unsuitable for consumption if the exposure time to ozone is not properly controlled.

Therefore, methods and apparatus for treating meat with ozone are in need of development. The present invention fulfills these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

One aspect of the invention is a method for sanitizing perishable goods by exposing the goods to ozone gas in an atomized solution for a suitable period of time followed by scrubbing of the ozone gas with other gases, such as air and then carbon dioxide, thereby substantially neutralizing any sanitizing agent with the goods.

The invention is directed to a method and apparatus for sanitizing perishable goods by exposing the goods to ozone for a suitable period of time followed by scrubbing the ozone gas with air and then carbon dioxide and substantially neutralizing any residual sanitizing agent left in the goods. Following neutralization, the goods may further be treated with an antioxidant to reduce the deleterious oxidizing effects of ozone on the goods. Goods can include meats, such as beef, lamb, veal, pork, chicken, and the like.

In another aspect of the present invention, a sanitizing apparatus for goods includes a horizontal conduit pressure vessel with a first section in which the goods are treated and a second scrubbing section. The pressure vessel encloses an Archimedes screw disposed on a horizontal, rotating axis, which carries and rotates the goods. The apparatus is configured to expose all surfaces of the goods to the gases contained within the pressure vessel, from the point of entry, through the first section of the vessel, the scrubbing section, and to the exit end of the pressure vessel. Any number of similar sections can process meat with differing agents, such as neutralizing fluids, gases, or antioxidants. Following the sanitizing step, the goods are ground (to ensure rapid adjustment of the pH level of the goods that may otherwise cause excessive oxidizing at the surface of the goods) and then selectively transferred and divided into at least two streams carried through corresponding conduits prior to subsequent proportioning and blending equipment.

The present invention can thus provide precise control of exposure time to concentrated ozone to a minimum, thus sanitizing the meat without causing deleterious effects on meat. A further advantage is the ability to keep the meat enclosed within a conduit and thus minimize exposure to atmospheric oxygen.

In one aspect of the invention, ozone and water are introduced into a vessel containing meat. Any amount of water can then be removed with a dry gas. The addition of water enhances the activity of ozone in beneficial ways for washing and sanitizing the meat.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows an illustration of an apparatus arranged to sanitize a continuous stream of boneless meat;

FIG. 2 shows an illustration of an apparatus arranged to sanitize a continuous stream of boneless meat; and

FIG. 3 shows an illustration of equipment arranged to produce ground meat after sanitizing with apparatus described in association with FIGS. 1 and 2.

### DETAILED DESCRIPTION

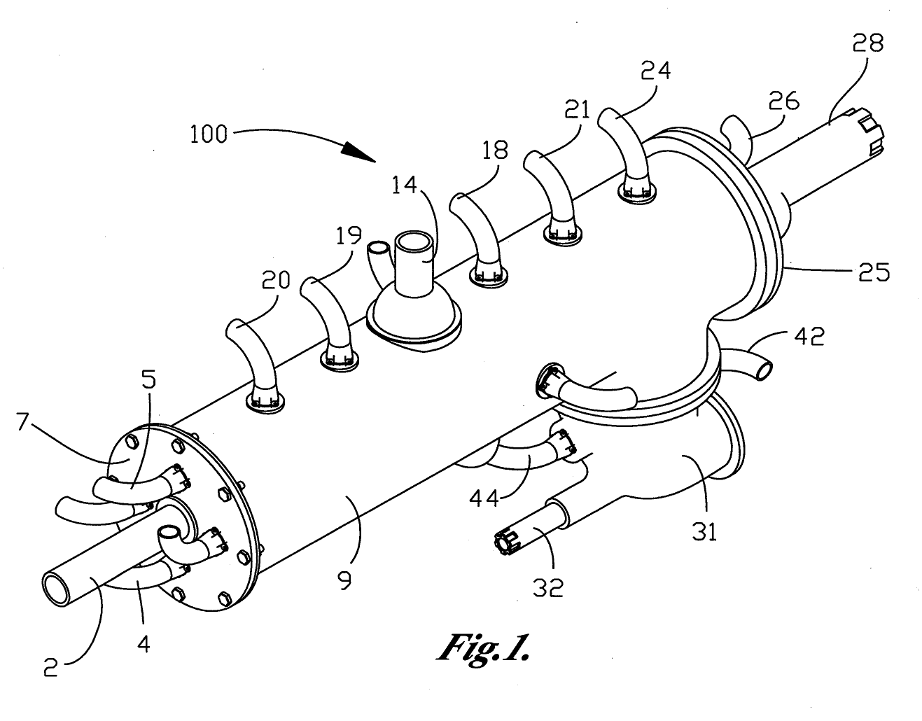

Referring now to FIG. 1, an apparatus 100 constructed for the purpose of sanitizing boneless meat is shown. The apparatus 100 includes a horizontally disposed conduit 9 with end caps 7 and 25 sealed thereto. End caps 7 and 25 are securely attached to conduit 9 and are removable to allow cleaning of the internal components of the apparatus 100. Port 2 is located centrally on the upstream side of the apparatus 100 in the end cap 7 and arranged to allow injection under pressure of boneless meat in a continuous stream therethrough, and into the pressure vessel 9. At the downstream end of the conduit 9, a driveshaft 28 is centrally located in end cap 25. A connection to pressure vessel 9 is located on the downstream underside of pressure vessel 9 to connect to a receptacle 31. A driveshaft 32 is mounted to receptacle 31. Gas injection ports 4, 20, and 24, for example, are provided and an exhaust port 14, is located centrally and on the upper side of pressure vessel 9.

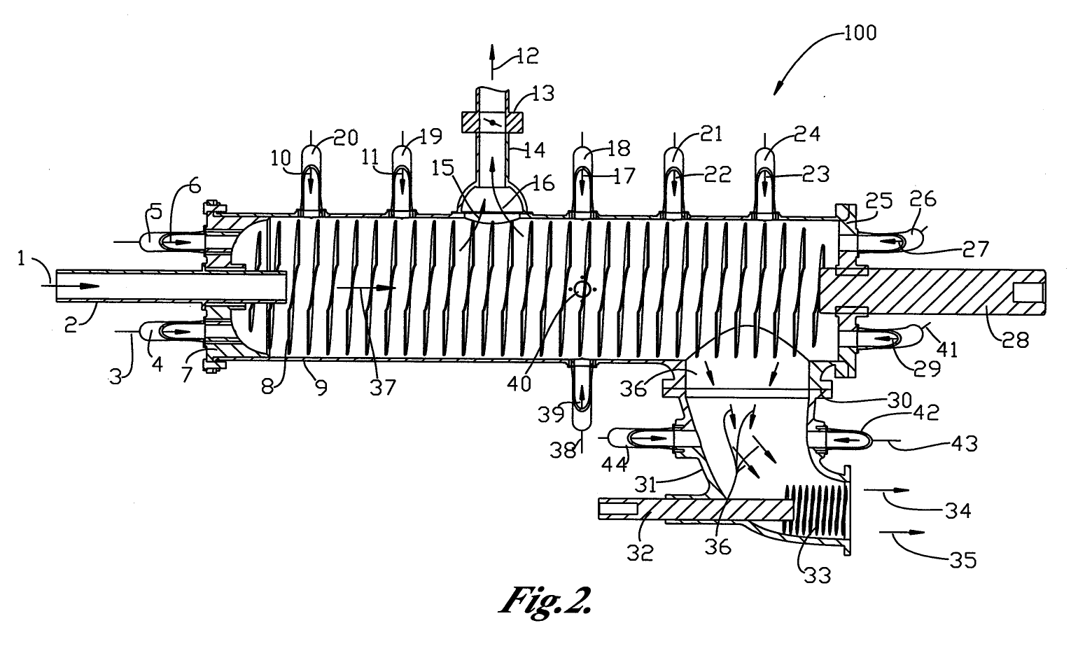

Referring now to FIG. 2, a cross section illustration through the apparatus 100 shown in FIG. 1 is provided. Port 2 allows the pressurized transfer of boneless meat in the direction shown by arrow 1, and into the pressure vessel 9. Boneless meat is transferred therein, under pressure, by pumping means such as with a meat pump manufactured by Marlen. Meat is transferred in the direction shown by arrow 1 continuously and at an adjustable mass flow rate. Endplate 7 is attached to pressure vessel 9, by suitable means such as bolts, and gas ports shown as 5 and 4 provide for the injection of selected gases, such as ozone or carbon dioxide, both of which may be in atomized solution form, wherein the quantity of ozone and water is injected at a variable and precise rate, and generally according to the mass flow of boneless meat transferred into the pressure vessel also. At the downstream end of pressure vessel 9, endplate 25 is securely attached and sealed, and is arranged with driveshaft 28 centrally disposed on endplate 25; driveshaft 28 is attached to Archimedes screw 8, which is located inside pressure vessel 9 in such a way that, when shaft 28 is rotated, the flights of Archimedes screw 8 rotate in close proximity to the internal surface of conduit 9 but do not touch the internal surface. As the screw 8 is rotated, boneless meat is transferred through pressure vessel 9. As the meat is transferred along a substantially horizontal path, boneless meat is rotated thereby exposing all surfaces of the meat to gas also injected into pressure vessel 9, thereby allowing for the killing of bacteria that may be present at the surfaces of meat by contact with the ozone solution and ozone gas. Ozone gas is injected at a suitable pressure that may be at 25 psi, or as high as 200 psi or more, and in volumes adequate to substantially ensure killing of surface bacteria on the meat. Ports 20 and 19 are also provided and ozone gas can be injected therethrough in the direction of arrows 10 and 11. Additionally, a precisely measured quantity of water may be injected via an atomizing injection device (not shown) directly into the pressure vessel 9. The water can be any amount. In some instances, the amount is any amount that exceeds the regulated allowable quantity of water in meat, for example. However, the water content in beef can be reduced with a gas as will be described below. The amount of water introduced can be metered and regulated. The water injection devices can be located adjacent the ozone injection ports 19 and 20, for example, such that ozone gas can dissolve into the atomized water. In this way, the dissolved ozone gas will provide a solution of ozonated water that can then contact the boneless meat's surfaces. Therefore, both ozone gas and ozonated water in an atomized condition will be present in the free spaces in the chamber of vessel 9 such that the gas can dissolve in the moisture on the meat's surfaces. Additionally, ozonated water will also be available to increase the quantity of ozone that contacts the meat's surface. In order to satisfy hazard analysis and critical control point (HACCP) requirements for meat decontamination processes, ozone measuring (monitoring) devices can be located at the point of ozone entry into the chamber 9 and even inside the ports such as 4 and 5, for example, so as to provide a reliable means of measuring the concentration of ozone gas at the entrance to vessel 9. An additional ozone measuring (monitoring) device(s) can be located at the point of gas exhaust from the chamber 9 and inside the exhaust port 14 so as to provide a reliable means of measuring the concentration of ozone gas at the point of exhaust and after it has been transferred through the chamber 9. In this way, when all relevant conditions such as temperature of the meat and the mass flow of the meat transferred through the chamber 9 are known and maintained within acceptable ranges, a reproducible process of decontaminating meat can be specified by controlling the quantity of ozone that is transferred into the chamber 9. A large proportion of the ozone gas that is provided into chamber 9 through ports, such as ports 4 and 5, will decompose into oxygen gas but a quantity may survive and therefore be exhausted via port 14. The bactericidal effectiveness of the ozone gas during its passage through the chamber 9 can be determined by the quantity of ozone gas that remains in the exhausted gases through port 14. In this way, a reproducible process of decontaminating meat that meets the HACCP standards, for example, can be developed and maintained. Ports 18, 38, and 40 are located immediately downstream from exhaust port 14, and a suitable gas that has, for example, been pretreated, such as by compressing, filtering and chilling, is injected therethrough in the direction of arrows 17 and 39 (arrow for port 40 not shown). Gas injected through ports 18, 38, and 40 can be chilled and dried thoroughly; and such gas may be filtered air, and injected at a pressure equal to other gases that are injected into pressure vessel 9, through other gas injection ports, such as ports 4 and 5. The purpose of injecting a dry gas, such as air, through ports 18, 38, and 40 is to dry and to reduce to a desired level the quantity of water that has been injected with ozone gas, through ports 5, 4, 20, and 19, for example. In some instances, the dry gas will reduce the amount of water in the vessel to produce a water content in the meat that is acceptable. The amount of dry gas can be metered and regulated in a specific amount. The dry gas injected through ports, 40, 38, and 18 will become saturated with water vapor, which will then be carried out of pressure vessel 9, through exhaust port 14, and in the direction of arrows 15 and 16. Ports 21, 24, 26, 41, 42, and 44 are provided to allow the injection of other selected gases, such as carbon dioxide, at a pressure equal to the injected pressure of other gases that are injected into pressure vessel 9. Carbon dioxide injected into these ports may be in atomized and chilled and be in solution form, where the carbon dioxide has been dissolved in water, under pressure, thereby producing carbonic acid that is then atomized prior to injection into pressure vessel 9. Such carbonic acid, which may have a pH of approximately 3.7, will provide additional sanitizing capability by killing bacteria that may have been injured by ozone injected upstream, or alternatively any bacteria that have escaped contact with ozone at the upstream end of pressure vessel 9. Exhaust port 14 is centrally located on the upper side of conduit 9, and has a pressure regulator 13 fitted thereto. Unused ozone gas, oxygen, moisture-laden air, and carbon dioxide or other gases will escape through exhaust port 14, at a preset pressure, such as 200 psi. However, the pressure within vessel 9 may be at more or less than 200 psi, and is most preferably at an optimum pressure that will maximize death of bacteria that may be present at the surface of the boneless meat being processed in the apparatus. Exhaust gas escapes in the direction shown by arrow 12, and may be vented to atmosphere or bubbled through water, and cleaned prior to exhausting to atmosphere. When used in the manner hereinabove described, the apparatus shown in FIG. 2 will not only process boneless meat at a controlled mass flow rate, and in doing so kill bacteria contained therein, it also provides a means of adjusting, with precise accuracy, the amount of water added to the boneless meat. For example, a mass flow of boneless meat equal to “x” pounds per hour, can be injected into pressure vessel 9, through port 2, and a quantity of water equal to 10% of “x,” for example, can be injected through gas injection ports, with the ozone gas and/or alternatively, with carbon dioxide gas. However, dry chilled air (or nitrogen gas), or any other suitable gas that is chilled and dried prior to injection, can be injected at such a rate that will vaporize the equivalent of half of the water transferred therein, and carry this vaporized water out of pressure vessel 9 through exhaust port 14. Therefore, in this way, a quantity of water equal to 5% of the volume of meat transferred therethrough, will be retained with the meat as it is transferred out of pressure vessel 9, and into receptacle 31 in the direction of arrows 36. To this end, injection ports for meat, ozone, dry gas or any other injection port can be fitted with a measuring instrument. A driveshaft 32, with Archimedes screw 33, is arranged to rotate and compress boneless meat into a single stream and directly into a coarse grinding plate, and in the direction of arrows 34 and 35. The rate of mass flow of coarse ground meat is equal to the rate of boneless meat transferred into pressure vessel 9 through conduit 2.

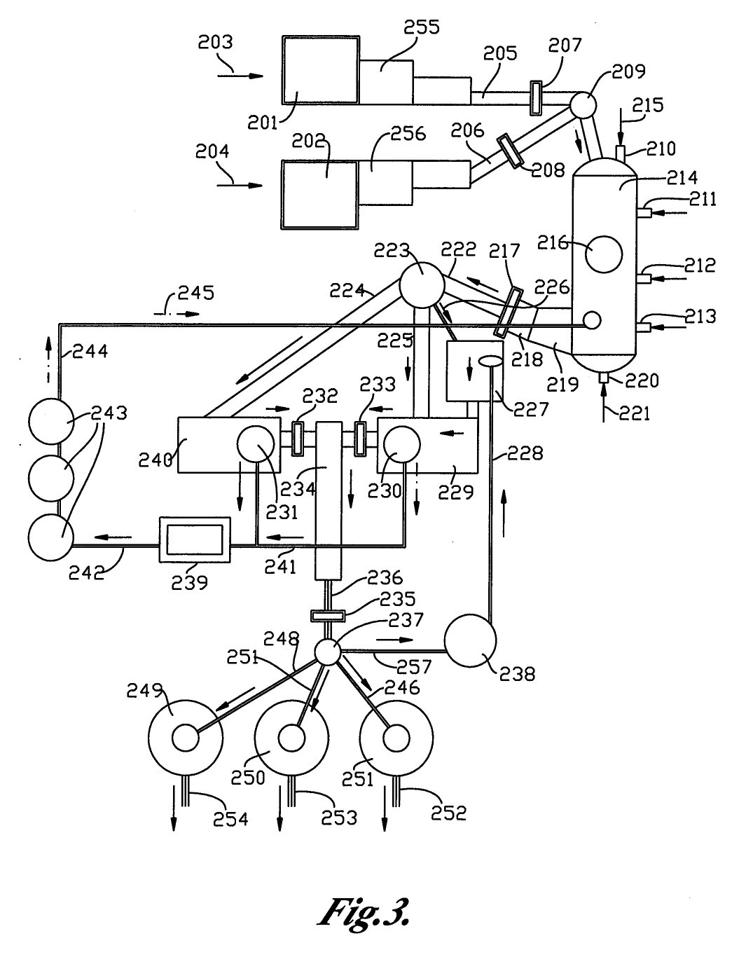

Referring now to FIG. 3, a plan view of a plant layout is illustrated. The equipment detailed in FIG. 3 is arranged to automatically sanitize or wash, grind, and proportion boneless meat with a selected lean to fat ratio. Combination dumpers 201 and 202 transfer boneless meat, in the direction shown by arrows 203 and 204, into conduits 205 and 206 via meat pumps 255 and 256. A supply of low fat content boneless meat is loaded at combination dumper 201, and a supply of relatively high fat content boneless meat is transferred in the direction of arrow 204, by dumper 202. Meat transferred in the direction shown by arrow 203, is pumped by pump 255, through conduit 205, and through x-ray fat measuring device 207, toward valve 209, and boneless meat is transferred in the direction of arrow 204, by pumping into conduit 206, by meat pump 256, through x-ray fat measuring device 208 and toward valve 209. Valve 209 is arranged to combine the two streams, or alternatively divert only meat from either stream 203 or stream 204, according to the measured fat content of each stream. Therefore, a single stream of meat is transferred directly into conduit 215, and transferred into meat processing apparatus 214, which is the apparatus described in connection with FIGS. 1 and 2 hereinabove. Gas injection ports 210, 211, 212, 213, and 220 allow selected gas injection in the direction of dotted arrows associated with each port. Processed boneless meat is then transferred through receptacle 219, into coarse meat grinder 218, and through x-ray fat measuring device 217, and into conduit 222. Conduit 222 is arranged to hold a predetermined quantity of boneless meat and is connected directly to diverter valve 223. Diverter valve 223 is arranged to directly transfer coarse ground meat from conduit 222, into any one of three conduits shown as 224, 225, or 226. The selection of any of the conduits 224, 225, and 226, is made according to the measured fat content continuously transferred through conduit 222, and according to the fat content measured by x-ray fat measuring device 217. In this way, a stream of boneless meat can be transferred along conduit 224, wherein the stream of meat has a relatively high level of fat. Alternatively, a stream of meat with a relatively low fat content can be transferred into conduit 225. In the event that any quantity of boneless meat has a level of fat content that is greater or lower than is acceptable, it can be transferred through conduit 226 into silo 227. Most preferably, the boneless meat stream directed through conduit 225 will be of relatively high fat content. The stream of meat transferred through conduit 224, is delivered into preblender 240, where it is blended and treated with liquid carbon dioxide. Similarly, boneless meat transferred along conduit 225 is delivered into preblender 229, where it is also blended and chilled with liquid carbon dioxide. Carbon dioxide gas is collected at locations 231 and 230, and tested for its purity. If testing shows that the carbon dioxide gas is substantially free of any other gases, such as atmospheric oxygen, it can be diverted to compressor 239 and stored in pressure vessels 243 until required for further use. Such further use may be in processing pressure vessel 214. In which case, the pressurized CO₂ gas can be transferred along conduits 244 and 245. Blended, coarse ground meat is transferred into continuous blender 234, from preblender 240, through x-ray fat measuring device 232, at a rate that is determined by the fat content as measured in x-ray device 232. Preblended boneless meat is transferred into continuous blender 234, from preblender 229, through x-ray fat measuring device 233, at a flow rate that is determined by fat content measured by device 233. A continuous single stream of blended boneless, coarse-ground meat is transferred from continuous blender 234, along conduit 236, through x-ray measuring device 235, and into diverter valve 237. Coarse-ground meat produced to specification is then diverted into either conduit 248, 247, 246, or 257, according to its measured fat content. Any such coarse-ground meat that does not meet specification, to the extent that its fat content is too high or too low, will be transferred into silo 238. Coarse-ground meat that has been produced according to requirements will be transferred into silos 249, 250, or 251, and retained therein, until required for further processing when the stored coarse-ground meat will be transferred from each silo, through respective conduits 254, 253, and 252. Any ground meat that does not meet specification and therefore has been transferred into silo 238 is then gradually transferred along conduit 228, into blender 227. Boneless meat that is stored in 227 is gradually transferred at a slow rate, into preblender 229. It should be noted that the entire apparatus shown in FIG. 3 has an atmosphere maintained within it that substantially eliminates the presence of oxygen gas, and is maintained at substantially 100% carbon dioxide. It should be noted that x-ray measuring devices 207, 208, 217, 232, 233, and 235, can be arranged to provide a sanitizing effect on the boneless meat that is transferred therethrough by elevating the intensity of x rays to the extent that bacteria is injured or killed as it passes therethrough. In this way, a systematic and gradual reduction in bacteria can be achieved, without the need for exposing the meat to a single source of x rays, sufficient to kill bacteria in a single step. X-ray measuring devices may also be configured to measure flow rate as well as any other meat attribute, including fat content.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for sanitizing perishable goods, comprising:
mixing the goods with a sanitizing agent for a suitable time followed by separating the agent in an enclosed conduit.

2. The method of claim 1, wherein the agent comprises ozone and water.

3. The method of claim 2, wherein the percent of ozone in water is about equal to or less than 16%.

4. The method of claim 1, further comprising compressing the goods to separate the sanitizing agent from the goods.

5. The method of claim 1, further comprising mixing the goods after exposure to the sanitizing agent with a neutralizing agent.

6. The method of claim 5, wherein the neutralizing agent includes chlorine dioxide.

7. The method of claim 5, further comprising a step of separating the neutralizing agent.

8. The method of claim 7, further comprising compressing the goods to separate the neutralizing agent.

9. The method of claim 1, further comprising mixing the goods after exposure to the sanitizing agent with an antioxidant agent.

10. The method of claim 9, wherein the antioxidant agent comprises citric acid.

11. A sanitizing apparatus for goods comprising:
means for mixing a good with a sanitizing agent; and
means for separating the agent from the goods.

12. The apparatus of claim 11, wherein the sanitizing apparatus includes a mixing chamber having paddles disposed on a rotating shaft, wherein paddles are spaced to assist separation of the sanitizing liquor from the goods.

13. The apparatus of claim 11, wherein the sanitizing apparatus includes a screw having a conical profile.

14. The apparatus of claim 11, further comprising means for mixing the goods with a neutralizing agent and means for separating the agent from the goods.

15. The apparatus of claim 14, further comprising a mixing chamber having paddles disposed on a rotating shaft, wherein paddles are spaced apart to separate the neutralizing agent from the goods.

16. The apparatus of claim 14, wherein the sanitizing apparatus includes a screw of increasing cylindrical diameter.

17. The apparatus of claim 14, further comprising means for mixing the goods with an antioxidant and means for separating the antioxidant from the goods.

18. The apparatus of claim 17, further comprising a mixing chamber having paddles disposed on a rotating shaft, wherein paddles are spaced to assist separation of an antioxidant from the goods.

19. The apparatus of claim 17, wherein the sanitizing apparatus includes a conical screw.

20. The apparatus of claim 14, further comprising a construction.

21. A method for processing meat, comprising:
adding ozone gas to meat; and
adding carbon dioxide to meat treated with ozone.

22. The method of claim 21, further comprising adding water to produce an ozonated solution in contact with the meat.

23. The method of claim 22, further comprising reducing water to a desired level by the introduction of a gas and removing said gas together with water.

24. The method of claim 22, wherein the addition of water is regulated according to the mass flow of meat.

25. The method of claim 21 wherein the addition of ozone is regulated by monitoring the amounts of ozone added or removed.

26. The method of claim 22, further comprising reducing the amount of water to produce a desired water content in meat.

27. A method for processing meat, comprising:
adding ozone gas to meat, followed by exposure to an acid.

28. The method of claim 27, wherein said acid is carbonic acid.

29. The method of claim 27, further comprising adding solubilized carbon dioxide gas under pressure.

30. A method for controlling water content in meat, comprising:
measuring the mass flow of meat;
regulating the addition of water according to the mass flow; and
removing water to produce meat with a desired content of water.

31. The method of claim 30, further comprising exposing said meat to a gas.

32. The method of claim 31, wherein the gas comprises carbon dioxide and a measured amount of carbon monoxide.
